(19) United States
(12) Patent Application Publication
(10) Pub. No.: US 2024/0203540 A1
(43) Pub. Date: Jun. 20, 2024

(54) SELF-SERVICE COHORT SELECTION FOR LARGE-SCALE OBSERVATIONAL STUDIES

(71) Applicants: City of Hope, Duarte, CA (US); The Regents of the University of California, Oakland, CA (US)

(72) Inventors: James V. Lacey, JR., Monrovia, CA (US); Jennifer Benbow, Monrovia, CA (US); Emma Spielfogel, Monrovia, CA (US); Kristen Savage, Monrovia, CA (US); Sandeep Chandra, San Diego, CA (US); Kai Lin, San Diego, CA (US)

(21) Appl. No.: 18/391,041

(22) Filed: Dec. 20, 2023

Related U.S. Application Data
(60) Provisional application No. 63/476,365, filed on Dec. 20, 2022.

(51) Int. Cl. G16H 10/20; G16H 10/60

(52) U.S. Cl. CPC G16H 10/20; G16H 10/60

(57) ABSTRACT

A method for self-service cohort selection may include receiving one or more user inputs specifying one or more cohort selection criteria. A script for accessing a first data store storing a first dataset may be generated based on the one or more cohort selection criteria. The script may be executed to retrieve, from the first dataset in the first data store, a subset of data. A second dataset corresponding to the first subset of data retrieved from the first data store may be generated for storage at the second data store. A visual representation of at least a portion of the second dataset may be generated for display at the client device. Related systems and computer program products are also provided.

### CROSS-REFERENCE TO RELATED APPLICATIONS

This application claims benefit of priority to U.S. Provisional Application No. 63/476,365, filed on Dec. 20, 2022, the contents of which is hereby incorporated by reference in its entirety.

STATEMENT AS TO FEDERALLY SPONSORED RESEARCH

This invention was made with government support under CA199277, and CA164917 awarded by the National Institutes of Health. The government has certain rights in the invention.

### TECHNICAL FIELD

The subject matter described herein relates generally to database processing and more specifically to self-service cohort selection for large-scale observational studies and disease registries.

### INTRODUCTION

Real-world data enables essential research on health and disease among communities. Large cohorts of research volunteers are designed to support a wide range of research projects. Cohort selection is the process of selecting a project-specific subset of data from a larger cohort. The huge volume of potential project-specific combinations of exposure data, endpoint data, and analytic designs makes cohort selection a challenge for providers and researchers.

### SUMMARY

Systems, methods, and articles of manufacture, including computer program products, are provided for self-service cohort selection. In one aspect, there is provided a system that includes at least one processor and at least one memory. The at least one memory may include program code that provides operations when executed by the at least one processor. The operations may include: receiving, from a first client device, a first user input specifying one or more cohort selection criteria; generating, based at least on the one or more cohort selection criteria, a script for accessing a first data store storing a first dataset; executing the script to retrieve, from the first dataset in the first data store, a first subset of data; generating, for storage at a second data store, a second dataset corresponding to the first subset of data retrieved from the first data store; and generating, for display at the first client device, a visual representation of at least a portion of the second dataset.

In another aspect, there is provided a method for self-service cohort selection. The method may include: receiving, from a first client device, a first user input specifying one or more cohort selection criteria; generating, based at least on the one or more cohort selection criteria, a script for accessing a first data store storing a first dataset; executing the script to retrieve, from the first dataset in the first data store, a first subset of data; generating, for storage at a second data store, a second dataset corresponding to the first subset of data retrieved from the first data store; and generating, for display at the first client device, a visual representation of at least a portion of the second dataset.

In another aspect, there is provided a computer program product including a non-transitory computer readable medium storing instructions. The instructions may cause operations when executed by at least one data processor. The operations may include: receiving, from a first client device, a first user input specifying one or more cohort selection criteria; generating, based at least on the one or more cohort selection criteria, a script for accessing a first data store storing a first dataset; executing the script to retrieve, from the first dataset in the first data store, a first subset of data; generating, for storage at a second data store, a second dataset corresponding to the first subset of data retrieved from the first data store; and generating, for display at the first client device, a visual representation of at least a portion of the second dataset.

In some variations of the methods, systems, and computer program products, one or more of the following features can optionally be included in any feasible combination.

In some variations, the first dataset may include a plurality of records. Each of the plurality of records may be associated with a participant.

In some variations, each of the plurality of records may be associated with a plurality of attributes corresponding to one or more exposures, genomic biomarkers, and/or clinical phenotypes of the participant.

In some variations, the plurality of attributes may include a date of birth, a race, an ethnicity, and a vital status of the participant.

In some variations, the plurality of attributes may include a first date when follow-up began, a second date when follow-up ended, and a third date of each follow-up survey.

In some variations, the plurality of attributes may include a site, a stage, a grade, and a diagnosis date for a disease associated with the participant.

In some variations, the script may be executed to identify, based on one or more of the plurality of attributes, one or more records matching the one or more cohort selection criteria.

In some variations, the script may be executed to identify, based on a combination of a first attribute and a second attribute from the plurality of attributes, one or more records matching the one or more cohort selection criteria.

In some variations, the combination of the first attribute and the second attribute may include a maximum, a minimum, a mean, a mode, a median, and/or a range of a respective values of the first attribute and the second attribute.

In some variations, the plurality of records may include a first record for a first disease associated with the participant and a second record for a second disease associated with the participant.

In some variations, the first dataset at the first data store may be preprocessed by at least identifying a first record and a second record of a same disease associated with the participant, and performing a deduplication that includes (i) removing the first record or the second record based on the first record and the second record being identical or (ii) combining the first record and the second record to generate a third record replacing the first record and the second record based on the first record and the second record each containing some but not all of the plurality of attributes.

In some variations, a user interface may be generated for receiving the first user input specifying the one or more cohort selection criteria, the user interface including a first input control for a first cohort selection criterion determined based on at least a portion of the plurality of attributes.

In some variations, the first input control may provide a selection between at least a first value and a second value for the first cohort selection criteria. The first value and the second value may be determined on at least the portion of the plurality of attributes.

In some variations, the user interface may further include a second input control for a second cohort selection criterion determined based on at least the portion of the plurality of attributes.

In some variations, the visual representation of at least the portion of the second dataset may include at least one of a heat map, a bar graph, a pie chart, and a line graph.

In some variations, the one or more cohort selection criteria may include an endpoint definition comprising one or more of a disease diagnosis, hospitalization, and mortality.

In some variations, the one or more cohort selection criteria may include one or more inclusion criteria or exclusion criteria.

In some variations, a second user input modifying the one or more cohort selection criteria may be received from the client device. The script for accessing the first data store storing the first dataset may be modified based at least on the one or more modified cohort selection criteria. The updated script may be executed to retrieve, from the first dataset in the first data store, a second subset of data. The second data store may be updated to include the second subset of data retrieved from the first data store.

In some variations, the second data store may be updated to include the first subset of data as a first version of the second dataset and the second subset of data as a second version of the second dataset.

In some variations, the second data store may be updated by at least replacing the first subset of data with the second subset of data as the second dataset.

In some variations, the first dataset may be generated by at least querying a third data store to retrieve at least a portion of a third dataset stored therein. The first data store may be updated to include the first dataset.

In some variations, the third data store may be a relational database and the first data store may be a non-relational database.

In some variations, the generating of the first dataset may include transforming at least a portion of the third dataset retrieved from the third data store from a predefined schema of the relational database to a dynamic schema of the non-relational database.

In some variations, the generating of the first dataset may include performing a domain based filtering of the third dataset.

In some variations, the generating of the first dataset may include joining at least the portion of the third dataset.

In some variations, the first user input specifying a first cohort selection criteria may be received from the first client device. A second user input modifying the first cohort selection criteria and/or specifying a second cohort selection criteria may be received from a second client device.

In some variation, in response to the first user input specifying a first value for a cohort selection criterion, the second dataset may be generated to correspond to the first dataset retrieved from the first data store. In response to the first user input specifying a second value for the cohort selection criterion, a further subset of the first subset of the first dataset may be generated to correspond to the second value of the cohort selection criterion and the second dataset may be generated to correspond to the further subset of the first subset of the first dataset.

In some variations, a user associated with the first client device may be authenticated by at least sending, to a project and user management system, a user credential information from an active directory of a secure environment and receiving, from the project and user management system, one or more client devices with access to a project associated with the first dataset.

Implementations of the current subject matter can include, but are not limited to, methods consistent with the descriptions provided herein as well as articles that comprise a tangibly embodied machine-readable medium operable to cause one or more machines (e.g., computers, etc.) to result in operations implementing one or more of the described features. Similarly, computer systems are also described that may include one or more processors and one or more memories coupled to the one or more processors. A memory, which can include a non-transitory computer-readable or machine-readable storage medium, may include, encode, store, or the like one or more programs that cause one or more processors to perform one or more of the operations described herein. Computer implemented methods consistent with one or more implementations of the current subject matter can be implemented by one or more data processors residing in a single computing system or multiple computing systems. Such multiple computing systems can be connected and can exchange data and/or commands or other instructions or the like via one or more connections, including, for example, to a connection over a network (e.g., the Internet, a wireless wide area network, a local area network, a wide area network, a wired network, or the like), via a direct connection between one or more of the multiple computing systems, etc.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes in relation to cohort selection in the context of cancer research, it should be readily understood that such features are not intended to be limiting. The claims that follow this disclosure are intended to define the scope of the protected subject matter.

### DESCRIPTION OF DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 depicts a system diagram illustrating an example of a cohort selection system, in accordance with some example embodiments;

FIG. 2 depicts a screenshot illustrating an example of a user interface, in accordance with some example embodiments;

FIG. 3 depicts a screenshot illustrating examples of visual representations of datasets, in accordance with some example embodiments;

FIG. 4 depicts a flowchart illustrating an example of a process for cohort selection, in accordance with some example embodiments; and

FIG. 5 depicts a block diagram illustrating an example of a computing system, in accordance with some example embodiments.

When practical, similar reference numbers denote similar structures, features, or elements.

### DETAILED DESCRIPTION

Observational research using real-world data makes vital and important contributions to research into various diseases such as cancer. Well-designed and rigorous cohort studies that enroll volunteers who agree to have their health data tracked and aggregated for future research are especially important for observational research. These cohorts transcend individual investigators or projects and become community resources that add value and economies of scale by enabling entire research communities to utilize a cohort's data and resources for hypothesis-driven research. The largest cohorts can include hundreds of thousands of participant partners (e.g., volunteers) and are designed to last for decades and support a wide range of future research.

Prospective cohorts collect exposure (e.g., surveys of patient-reported outcomes or lifestyle factors) and clinical phenotype (e.g., via linkage with electronic health records or administrative claims data) data that can be used in various analytic designs, including time-to-event analyses, nested case-control studies, or cross-sectional comparisons. Even with modest sample sizes, the number of potential projects (e.g., all possible combinations of analytic designs, exposures, and phenotypes) in a typical cohort is enormous. Adding genomic or other biomarker data creates even more potential possibilities. For example, the 500,000-participant UK Biobank, a relatively new cohort, can already support tens of millions of potential projects.

Projects rarely if ever require all the data a cohort possesses, and cohorts never provide all of their data to individual researchers or research projects. Instead, researchers need project-specific subsets of a cohort's data. In this context, the term “cohort selection” refers to the process of selecting and providing custom data for every research project. Cohorts approach cohort selection in different ways, but the process typically involves applying inclusion & exclusion criteria, defining the parameters of a specific analysis, and choosing the specific covariates and endpoint data needed for that analysis.

Cohort selection is often a bottlenecking event. For cohorts that keep their data on premise (e.g., behind institutional firewalls), cohort selection can require extensive back-and-forth conversations between investigators and cohorts with the cohort team then applying those decisions to generate custom datasets that are made available to investigators. Newer cohorts and networks, such as the UK Biobank and CRDN, use cloud resources to make their data available, but cohort selection can still take weeks or months and require significant resources from both cohorts and investigators. In some example embodiments, a cohort selection engine may improve cohort-selection by supporting a direct configuration of project specific design and data. The cohort selection engine may include a self-service cohort-selection tool capable of accommodating an extensive range of potential combinations of design and data within a large prospective cohort.

In some example embodiments, the cohort selection engine may receive, from a client device, a user input specifying one or more cohort selection criteria. The cohort selection engine may generate, based at least on the one or more cohort selection criteria, a script for accessing a first data store storing a first dataset. The cohort selection engine may execute the script to retrieve, from the first dataset in the first data store, a first subset of data before generating, for storage at a second data store, a second dataset (e.g., a custom dataset) corresponding to the first subset of data retrieved from the first data store. Moreover, in some cases, the cohort selection engine may generate one or more custom documentation and scripts that enable the consumption of the second dataset stored at the second data store. While no two cohorts are identical, large-scale cohorts do share common design and data features that make cohort-selection a universal challenge. The cohort selection engine described herein may improve cohort-selection particularly within large-scale research resources that share their data.

In some example embodiments, the cohort selection engine may generate the first dataset by querying a third data store to retrieve at least a portion of a third dataset stored therein before updating the first data store to include the first dataset. For example, in some cases, the cohort selection engine may generate the first dataset by performing a domain based filtering of the third dataset stored in the third data store. Alternatively and/or additionally, the cohort selection engine may generate the first dataset by joining at least a portion of the third dataset. In some cases, the first data store may be a non-relational (or NoSQL) database whereas the third data store may be a relational (or SQL) database. Accordingly, the generating of the first dataset may include transforming at least a portion of the third dataset retrieved from the third data store from a predefined schema of the relational database to a dynamic schema of the non-relational database.

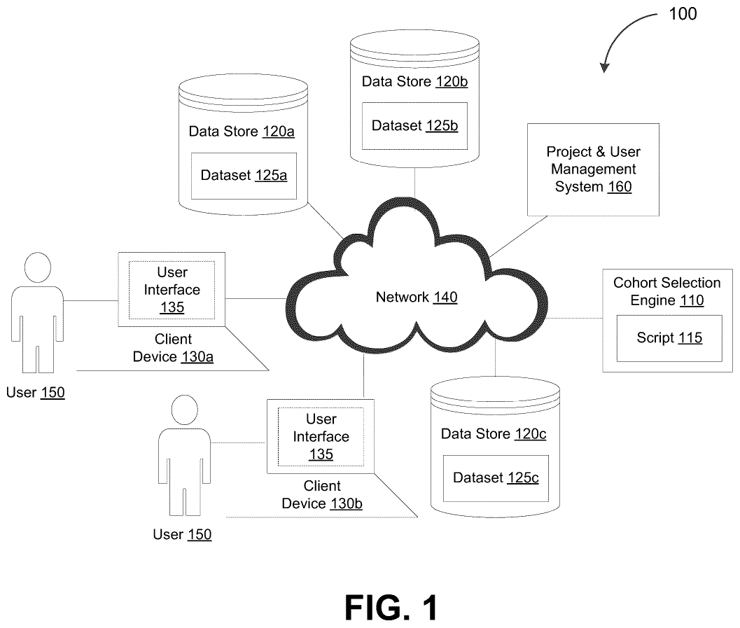

FIG. 1 depicts a system diagram illustrating an example of a cohort selection system 100, in accordance with some example embodiments. Referring to FIG. 1, the cohort selection system 100 may include a cohort selection engine 110, one or more data stores 120, and one or more client devices 130. As shown in FIG. 1, the cohort selection engine 110, the one or more data stores 120, and the one or more client devices 130 may be communicatively coupled via a network 140. The one or more client devices 130 may be a processor-based device including, for example, a workstation, a desktop computer, a laptop computer, a smartphone, a tablet computer, a wearable apparatus, and/or the like. The network 140 may be a wired network and/or a wireless network including, for example, a local area network (LAN), a virtual local area network (VLAN), a wide area network (WAN), a public land mobile network (PLMN), the Internet, and/or the like.

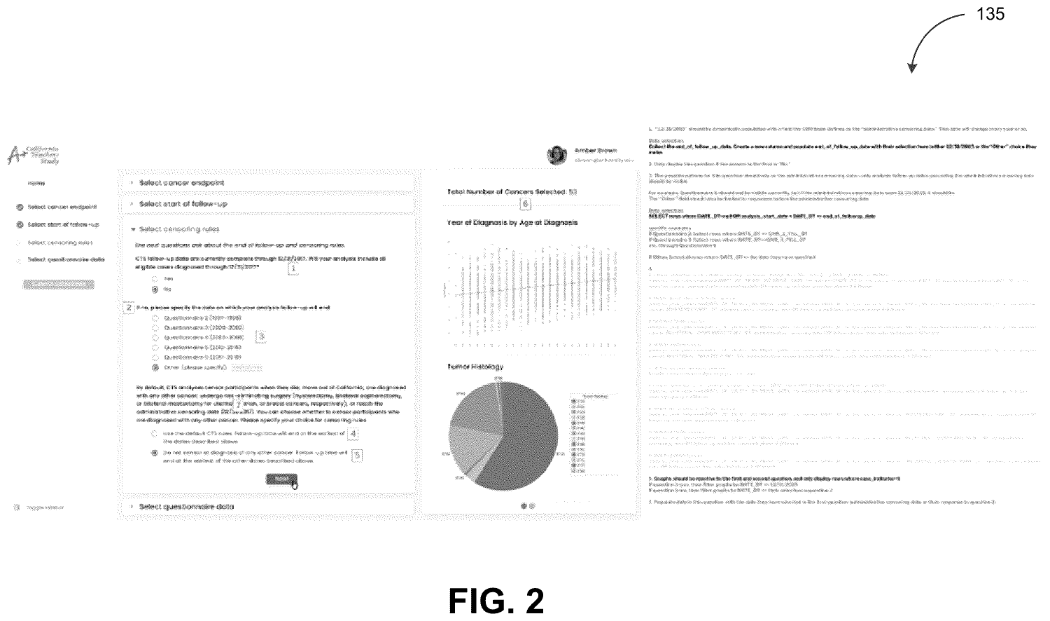

Referring to FIG. 1, the one or more client devices 130 may include a user interface 135 configured to receive one or more user inputs from a user 150 at the one or more client devices 130. For example, in some cases, the user interface 135 may be a part of a web-based application associated with the cohort selection engine 110. As shown in FIG. 1, the cohort selection engine 110 may receive, from a first client device 130a, a first user input specifying a first cohort selection criteria. Moreover, in some cases, the cohort selection engine 110 may support collaboration across multiple client devices 130. Accordingly, in addition to the first user input received from the first client device 130a, the cohort selection engine 110 may also receive, from a second client device 130b, a second user input modifying the first cohort selection criteria and/or specifying a second cohort selection criteria. The cohort selection engine 110 may generate, based at least on the one or more cohort selection criteria, a script 115 for accessing a first data store 120a storing a first dataset 125a. The cohort selection engine 110 may execute the script 115 to retrieve, from the first dataset 125a in the first data store 120a, a first subset of data before generating, for storage at a second data store 120b, a second dataset 125b corresponding to the first subset of data retrieved from the first data store 120a. In some example embodiments, the cohort selection engine 110 may generate the user interface 135 to display, at the client device 130, a visual representation of at least a portion of the second dataset 125b stored at the second data store 120b. As shown in FIG. 2, examples of the visual representation in this context may include a heatmap, a bar graph, a pie chart, a line graph, and/or the like. Moreover, in some cases, the cohort selection engine 110 may generate one or more custom documentation and additional scripts that enable the consumption of the second dataset 125b stored at the second data store 120b.

For example, in some cases, once the cohort-selection process is complete, the cohort selection engine 110 may save the user inputs received from the one or more client devices 130. Moreover, the cohort selection engine 110 may generate a variety of deliverables including 1) a custom dataset in a *.csv format, for consumption by open-source software; 2) an identical version of that dataset but formatted for use in an analysis system such as STATISTICAL ANALYSIS SYSTEM (SAS), which is the primary software used in the cancer epidemiology cohort (CEC) community; 3) an analysis system-specific formats file to accompany the analysis system dataset; 4) a custom data dictionary, based on the presentation database master dictionary, that includes all of the covariates selected (and omits all covariates that were not selected); and 5) a summary of all of the cohort-selection design choices, such as start-of-follow-up, end-of-follow-up, and specific cancer sites and histologic codes that were included in the cancer outcome. The cohort selection engine 110 may write deliverables automatically to a read-only project-specific directory within, for example, a remote desktop environment at the one or more client devices 130. Doing so may provide the user 150 with the data and documentation necessary for downstream analyses. New code and/or scripts may be created within user-specific project folders within the remote desktop environment. Writing the data to a read-only drive may facilitate data governance and enable version control. In addition, this strategy may preserve data fidelity from these output datasets back to the second data store 120b (e.g., the presentation database) and ultimately the first data store 120a and/or the third data store 120c. The cohort selection engine 110 may be capable of generating output files in very little time, providing essentially immediate access to any necessary data, tools, and documentation.

In some example embodiments, the cohort selection engine 110 may receive, from the client device 130, a second user input modifying the one or more cohort selection criteria. The cohort selection engine 110 may respond to the receiving the second user input by at least updating, based at least on the one or more modified cohort selection criteria, the script 115 for accessing the first data store 120a storing the first dataset 125a. The cohort selection engine 110 may execute the updated script 115 to retrieve from the first dataset 125a stored at the first data store 120a, a second subset of data before updating the second data store 120b to include the second subset of data retrieved from the first data store 120a. For example, in some cases, the second data store 120b may be updated to include the first subset of data as a first version of the second dataset 125b and the second subset of data as a second version of the second dataset 125b. Alternatively, the second data store 120b may be updated by replacing the first subset of data with the second subset of data as the second dataset 125b.

In some example embodiments, the cohort selection engine 110 may generate the first dataset 125a by at least querying a third data store 120c to retrieve at least a portion of a third dataset 125c stored therein before updating the first data store 120a to include the first dataset 125a. For example, in some cases, the query selection engine 110 may generate the first dataset 125a by performing a domain based filtering of the third dataset 125c stored at the third data store 120c. Alternatively and/or additionally, the cohort selection engine 110 may generate the first dataset 125a by joining at least the portion of the third dataset 125c stored at the third data store 120c. In some cases, the first data store 120a may be a non-relational (or NoSQL) database whereas the third data store 120c may be a relational (or SQL) database. Accordingly, in some instances, the generating of the first dataset 125a may further include transforming at least a portion of the third dataset 125c retrieved from the third data store 120c from a predefined schema of the relational database to a dynamic schema of the non-relational database.

Follow-up data updating the third dataset 125c at the third data store 120c, and consequently the first dataset 125a at the first data store 120a and the second dataset 125b at the second data store 120b may undergo regular updates (e.g., annual updates). Accordingly, in some example embodiments, the second data store 120b may be a versioned presentation database and the second dataset 125b a portion of the first dataset 125a extracted from the first data store 120a. The second data store 120b may therefore serve as an intermediary between the one or more client devices 130 and the underlying first data store 120a and the third data store 120c such that neither the first dataset 125a nor the third dataset 125c are directly accessible.

In some cases, the third dataset 125c may include so-called missing-by-design data. Nevertheless, as large prospective studies follow their participants for years and decades, the combination of study censoring (e.g., participants who die do not complete subsequent follow-up surveys) and rare but important outcomes (e.g., small percentages of participants have second, third, and fourth primary cancers) create valuable data in subsets of the study population. As such, while the third data store 120c is implemented as a relational (or SQL) database, the second data store 120b may be implemented as a non-relational (or NoSQL) database. Serving as the presentation database for data selected from the third data store 120c, implementing the second data store 120b as a non-relational (or NoSQL) database may increase the flexibility, scalability, and speed of the cohort selection engine 110.

As the presentation database, the second data store 120c may be populated with key data from three main domains including participant, outcome, and exposure. Participant data may include various characteristics (e.g., date of birth, race/ethnicity, vital status, and/or the like) as well as follow-up information (e.g., date follow-up began, date follow-up ended, dates of follow-up surveys, and/or the like). In the case of cancer, outcome data may include detailed disease information (e.g., site, stage, grade, diagnosis date, and/or the like) for all cancers during follow-up. Exposure data may include approximately 6000 covariates (i.e., columns) from the baseline as well as follow-up surveys. These data included at least one column for every question that was asked, plus numerous existing covariates derived from those responses (e.g., calculated body mass index (BMI) based on self-reported height and weight). The questionnaire covariates may be tagged to facilitate identification by question number, questionnaire section, or questionnaire number.

In some example embodiments, the first dataset 125a stored at the first data store 120a may include a plurality of records, each of which being associated with a participant. For example, in some cases, each record in the first dataset 125a may include a plurality of attributes of the corresponding participant. Examples of attributes in this context may include one or more exposures, genomic biomarkers, clinical phenotypes, a date of birth, a race, an ethnicity, a vital status, a first date when follow-up began, a second date when follow-up ended, a third date of each follow-up survey, a site of a disease, a stage of a disease, a grade of a disease, and a diagnosis date of a disease. In some cases, the records in the first dataset 125a may include analytic outcomes (e.g., International Classification of Diseases (ICD) based outcomes such as cancer, mortality, or hospitalization) as well as various types of exposure data (e.g., self-report from surveys, geospatial, or biospecimens). It should be appreciated that exposures and outcomes tend to be covariates with exposures being independent variables and analytic outcomes being dependent variables. The cohort selection engine 110 described herein may be configured to support cohort designs with any combination of analytic outcomes and exposure data (e.g., cancer as the analytic outcome and self-reported survey data as the exposures), including combinations of multiple outcomes and multiple exposures. For example, in some cases, the covariates may be selected individually or by hierarchical categories. Moreover, the cohort selection engine 110 may generate the first subset of the first dataset 125a by applying different start-of-follow-up, end-of-follow-up, and analytic censoring rules.

In some example embodiments, the cohort selection engine 110 may execute the script 115 to identify, based on a combination of a first attribute and a second attribute from the aforementioned plurality of attributes, one or more records in the first dataset 125a matching the one or more cohort selection criteria. In some cases, the combination of the first attribute and the second attribute may include a maximum, a minimum, a mean, a mode, a median, and/or a range of a respective values of the first attribute and the second attribute. Moreover, in some cases, the plurality of records included in the first dataset 125a may include, for a single participle, multiple records of associated with a same disease or different diseases. For example, the plurality of records included in the first dataset 125a may include a first record for a first disease associated with a participant and a second record for the first disease or a second disease associated with the same participant. Accordingly, in some instances, the cohort selection engine 110 may preprocess the first dataset 125a at the first data store 120a by at least identifying a first record and a second record of a same disease associated with the participant, and performing a deduplication that includes (i) removing the first record or the second record based on the first record and the second record being identical or (ii) combining the first record and the second record to generate a third record replacing the first record and the second record based on the first record and the second record each containing some but not all of the plurality of attributes.

As noted, in some cases, the user interface 135 at the client device 130 may be configured to receive one or more user inputs from the user 150 at the one or more client devices 130. A screenshot depicting an example of the user interface 135 is shown in FIG. 2. In some example embodiments, the cohort selection engine 110 may generate the user interface 135 to include one or more input controls including, for example, a first input control for a first cohort selection criterion, a second input control for a second cohort selection criterion, and/or the like. In some cases, the user interface 135 may include a series of drop-down menus and search functions to facilitate a selection amongst, for example, different cancer sites, cancer histologies, or related survey covariates within a particular survey. The cohort selection engine 110 may be configured to interim progress (e.g., locally at the one or more client devices 130, remotely at the cohort selection engine 110, and/or the like), thus subsequent edits, including back-and-forth navigation across different cohort selection criteria, and collaboration across multiple users 150 and/or multiple client devices 130.

The first cohort selection criterion and the second cohort selection criterion may each be determined based on at least a portion of the aforementioned plurality of attributes. Examples of the cohort selection criteria may include various inclusion criteria and/or exclusion criteria. For instance, in some cases, the first cohort selection criterion and/or the second cohort selection criterion may include an endpoint definition such as one or more of a disease diagnosis, hospitalization, and mortality. In some cases, the input control associated with a particular cohort selection criterion may provide a selection between multiple values such as a first value, a second value, and/or the like. In some cases, the selection of values associated with a cohort selection criterion may also be determined based on at least the portion of the plurality of attributes.

In some example embodiments, the cohort selection engine 110 may generate a further subset of the first dataset 125a when generating the second dataset 125b when a first value associated with a cohort selection criterion is selected instead of a second value associated with the cohort selection criterion. For example, for a cohort selection criterion that includes an endpoint definition, the cohort selection engine 110 may generate the second dataset 125b to correspond to the first subset of the first dataset 125a when a particular disease diagnosis (e.g., cancer) is selected as the endpoint definition. In doing so, the second dataset 125b may include records of participants who are documented as having been diagnosed with the particular disease (e.g., cancer). Alternatively, where hospitalization or mortality is selected as the endpoint definition, the cohort selection engine 110 may generate the second dataset 125 to correspond to a further subset of the first dataset 125a (e.g., a subset of the first subset of the first dataset 125a) such that the second dataset 125 includes records of participants who have been diagnosed with the particular disease (e.g., cancer) but who have further undergone hospitalization or have passed away.

Referring again to FIG. 1, in some cases, the cohort selection engine 110 may be linked via an integration to a project & user management system 160. In some cases, the link between the cohort selection engine 110 and the project and user management system 160 may be bidirectional. Accordingly, when the user 150 at the one or more client devices 130 attempts a login, the cohort selection engine 110 may send, to the project and user management system 160, user credential information from an active directory of a secure environment associated with the cohort selection engine 110. In response, the project and user management system 160 may return, to the cohort selection engine 110, information about a corresponding cohort selection project including, for example, the disease endpoints, the clients with access to the cohort selection project, and/or the like.

In cases where a project requires additional data joins of the underlying first dataset 125a (at the first data store 120a) and/or the third dataset 125c (at the third data store 120c) that are not in the second data store 120b (e.g., the presentation database), existing templates may be modified to deposit those data excerpts into a project team specific read-only directory. In some cases, the data joins may be achieved through a universal data key, thus providing essentially immediate access to these additional or custom data.

To facilitate cohort selection, the cohort selection engine 110 may automatically provide one or more covariates that are essential (e.g., dates of birth, death, and baseline survey) or have been observed in a more than a threshold quantity of analyses (e.g., body mass index (BMI), smoking status, and/or the like). Instead of requiring every decision to be made from scratch, the cohort selection engine 110 may generate the user interface 135 to provide certain default choices on key analytic decisions, such as excluding participants with prevalent cancers, while also allowing researchers to make alternative choices.

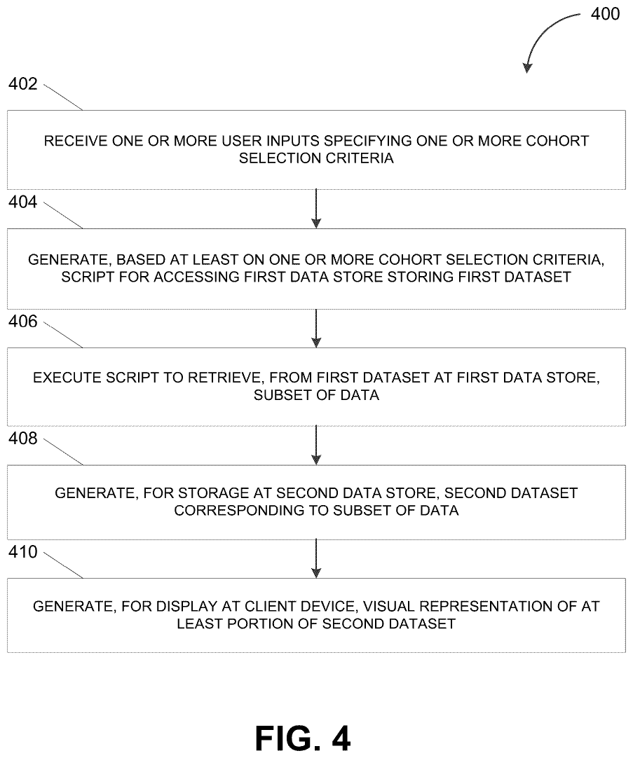

FIG. 4 depicts a flowchart illustrating an example of a process 400 for cohort selection, in accordance with some example embodiments. Referring to FIGS. 1 and 4, the process 400 may be performed by the cohort selection engine 110 in order to populate the second data store 120b (e.g., the presentation database) with the second dataset 125b, which corresponds to at least a portion of the first dataset 125a at the first data store 120a.

At 402, the cohort selection engine 110 may receive one or more user inputs specifying one or more cohort selection criteria. In some example embodiments, the cohort selection engine 110 may receive, from the first client device 130a, a first user input specifying a first cohort selection criteria. In some cases, the cohort selection engine 110 may further receive, from the second client device 130b, a second user input modifying the first cohort selection criteria and/or specifying a second cohort selection criteria. In some cases, the cohort selection engine 110 may generate the user interface 135, which may be displayed at the first client device 130a and/or the second client device 130b to receive the one or more user inputs. For example, in some cases, the cohort selection engine 110 may generate the user interface 135 to include a first input control for receiving the first user input specifying the first cohort selection criterion and a second input control for receiving the second user input specifying the second cohort selection criterion. In some cases, the first input control and/or the second input control may provide a selection between multiple values including, for example, a first value, a second value, and/or the like. In some cases, the cohort selection criteria included in the user interface 135 and the selection of values associated with each cohort selection criteria may be determined based on a plurality of attributes associated with each of the records included in the first dataset 125a. As noted, examples of attributes may include one or more exposures, genomic biomarkers, clinical phenotypes, a date of birth, a race, an ethnicity, a vital status, a first date when follow-up began, a second date when follow-up ended, a third date of each follow-up survey, a site of a disease, a stage of a disease, a grade of a disease, and a diagnosis date of a disease.

At 404, the cohort selection engine 110 may generate, based at least on the one or more cohort selection criteria, a script for accessing a first data store storing a first dataset. In some example embodiments, the cohort selection engine 110 may generate, based at least on the one or more cohort selection criteria, the script 115. In some cases, the script 115 may be configured to retrieve, from the first dataset 125a in the first data store 120a, a first subset of data that is consistent with the first cohort selection criterion and/or the second cohort selection criterion specified by the one or more user inputs received from the one or more client devices 130. In some cases, in addition to the script 115, the cohort selection engine 110 may also generate one or more custom documentation and additional scripts that enable the consumption of the second dataset 125b stored at the second data store 120b.

At 406, the cohort selection engine 110 may execute the script to retrieve, from the first dataset at the first data store, a subset of data. For example, in some example embodiments, the cohort selection engine 110 may execute the script 115 to retrieve, from the first dataset 125a at the first data store 120a, a first subset of data. In some cases, depending on the values selected for the first cohort selection criterion and/or the second cohort selection criterion, a further subset of the first subset of data may be retrieved from the first dataset 125a at the first data store 120a. For instance, for a cohort selection criterion that includes an endpoint definition, the cohort selection engine 110 may execute the script 115 to retrieve the first subset of data from the first dataset 125a stored at the first data store 120a where a first value is selected for the cohort selection criterion (e.g., cancer instead of mortality or hospitalization). Alternatively, where a second value is selected for the cohort selection criterion (e.g., mortality or hospitalization instead of cancer), the cohort selection engine 110 may execute the script 115 to retrieve a further subset of the first subset of data from the first dataset 125a.

At 408, the cohort selection engine 110 may generate, for storage at a second data store, a second dataset corresponding to the subset of data. In some example embodiments, the cohort selection engine 110 may generate, for storage at the second data store 120b, the second dataset 125b to correspond to either the first subset of data (or the further subset of the first subset of data) from the first dataset 125a stored at the first data store 120a. In some cases, after the second dataset 125b has been generated for storage at the second data store 120b, the cohort selection engine 110 may receive, from the one or more client devices 130, a third user input modifying the one or more cohort selection criteria. Accordingly, the cohort selection engine 110 may update, based at least on the one or more modified cohort selection criteria, the script 115 for accessing the first data store 120a storing the first dataset 125a. Moreover, the cohort selection engine 110 may execute the updated script 115 to retrieve, from the first dataset 125a stored at the first data store 120a, a second subset of data. In some cases, the cohort selection engine 110 may update the second data store 120b to include the first subset of data as a first version of the second dataset 125b and the second subset of data as a second version of the second dataset 125b. Alternatively, the second data store 120b may be updated by replacing the first subset of data with the second subset of data as the second dataset 125b.

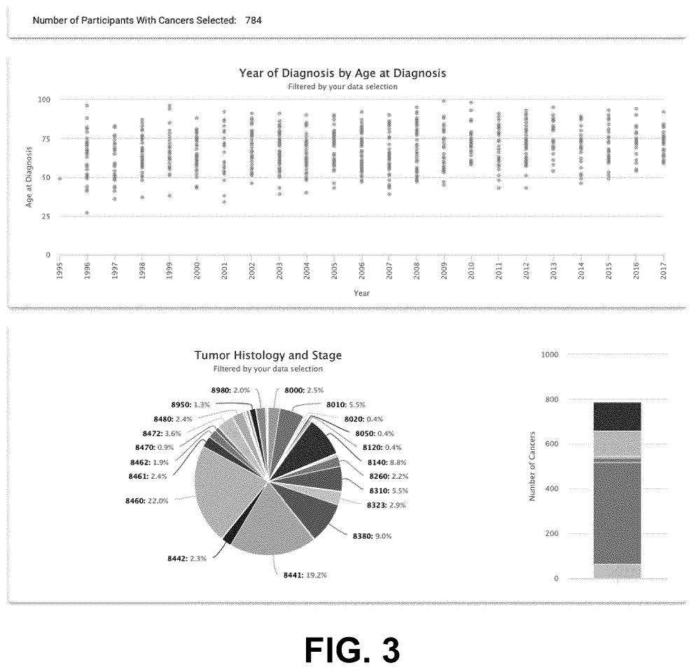

At 410, the cohort selection engine 110 may generate, for display at the client device, a visual representation of at least a portion of the second dataset. In some example embodiments, the cohort selection engine 110 may generate (or update) the user interface 135 to display, at the one or more client device 130, a visual representation of at least a portion of the second dataset 125b stored at the second data store 120b. As shown in FIG. 3, in some cases, the visual representation of at least the portion of the second dataset 125b may include one or more of a heatmap, a bar graph, a pie chart, a line graph, and/or the like.

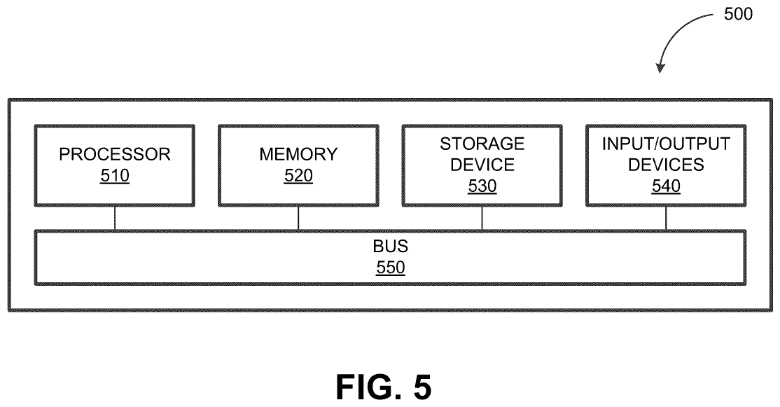

FIG. 5 depicts a block diagram illustrating an example of computing system 500, in accordance with some example embodiments. Referring to FIGS. 1 and 5, the computing system 500 may be used to implement the cohort selection engine 110, the one or more client devices 130, the project and user management system 160, and/or any components therein.

As shown in FIG. 5, the computing system 500 can include a processor 510, a memory 520, a storage device 530, and input/output devices 540. The processor 510, the memory 520, the storage device 530, and the input/output devices 540 can be interconnected via a system bus 550. The processor 510 is capable of processing instructions for execution within the computing system 500. Such executed instructions can implement one or more components of, for example, the cohort selection engine 110, the one or more client devices 130, the project and user management system 160, and/or the like. In some example embodiments, the processor 510 can be a single-threaded processor. Alternately, the processor 510 can be a multi-threaded processor. The processor 510 is capable of processing instructions stored in the memory 520 and/or on the storage device 530 to display graphical information for a user interface provided via the input/output device 540.

The memory 520 is a computer readable medium such as volatile or non-volatile that stores information within the computing system 500. The memory 520 can store data structures representing configuration object databases, for example. The storage device 530 is capable of providing persistent storage for the computing system 500. The storage device 530 can be a floppy disk device, a hard disk device, an optical disk device, or a tape device, or other suitable persistent storage means. The input/output device 540 provides input/output operations for the computing system 500. In some example embodiments, the input/output device 540 includes a keyboard and/or pointing device. In various implementations, the input/output device 540 includes a display unit for displaying graphical user interfaces.

According to some example embodiments, the input/output device 540 can provide input/output operations for a network device. For example, the input/output device 540 can include Ethernet ports or other networking ports to communicate with one or more wired and/or wireless networks (e.g., a local area network (LAN), a wide area network (WAN), the Internet).

In some example embodiments, the computing system 500 can be used to execute various interactive computer software applications that can be used for organization, analysis and/or storage of data in various formats. Alternatively, the computing system 500 can be used to execute any type of software applications. These applications can be used to perform various functionalities, e.g., planning functionalities (e.g., generating, managing, editing of spreadsheet documents, word processing documents, and/or any other objects, etc.), computing functionalities, communications functionalities, etc. The applications can include various add-in functionalities or can be standalone computing products and/or functionalities. Upon activation within the applications, the functionalities can be used to generate the user interface provided via the input/output device 540. The user interface can be generated and presented to a user by the computing system 500 (e.g., on a computer screen monitor, etc.).

One or more aspects or features of the subject matter described herein can be realized in digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including acoustic, speech, or tactile input. Other possible input devices include touch screens or other touch-sensitive devices such as single or multi-point resistive or capacitive track pads, voice recognition hardware and software, optical scanners, optical pointers, digital image capture devices and associated interpretation software, and the like.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

What is claimed is:

1. A computer-implemented method, comprising:
receiving, from a first client device, a first user input specifying one or more cohort selection criteria;
generating, based at least on the one or more cohort selection criteria, a script for accessing a first data store storing a first dataset;
executing the script to retrieve, from the first dataset in the first data store, a first subset of data;
generating, for storage at a second data store, a second dataset corresponding to the first subset of data retrieved from the first data store; and
generating, for display at the first client device, a visual representation of at least a portion of the second dataset.

2. The method of claim 1, wherein the first dataset includes a plurality of records, and wherein each of the plurality of records is associated with a participant.

3. The method of claim 2, wherein each of the plurality of records is associated with a plurality of attributes corresponding to one or more exposures, genomic biomarkers, and/or clinical phenotypes of the participant.

4. The method of claim 3, wherein the plurality of attributes include a date of birth, a race, an ethnicity, and a vital status of the participant.

5. The method of claim 3, wherein the plurality of attributes include a first date when follow-up began, a second date when follow-up ended, and a third date of each follow-up survey.

6. The method of claim 3, wherein the plurality of attributes include a site, a stage, a grade, and a diagnosis date for a disease associated with the participant.

7. The method of claim 3, wherein the script is executed to identify, based on one or more of the plurality of attributes, one or more records matching the one or more cohort selection criteria.

8. The method of claim 3, wherein the script is executed to identify, based on a combination of a first attribute and a second attribute from the plurality of attributes, one or more records matching the one or more cohort selection criteria.

9. The method of claim 8, wherein the combination of the first attribute and the second attribute comprises a maximum, a minimum, a mean, a mode, a median, and/or a range of a respective values of the first attribute and the second attribute.

10. The method of claim 2, wherein the plurality of records include a first record for a first disease associated with the participant and a second record for a second disease associated with the participant.

11. The method of claim 2, further comprising:
preprocessing the first dataset at the first data store by at least identifying a first record and a second record of a same disease associated with the participant, and performing a deduplication that includes (i) removing the first record or the second record based on the first record and the second record being identical or (ii) combining the first record and the second record to generate a third record replacing the first record and the second record based on the first record and the second record each containing some but not all of the plurality of attributes.

12. The method of claim 2, further comprising:
generating a user interface for receiving the first user input specifying the one or more cohort selection criteria, the user interface including a first input control for a first cohort selection criterion determined based on at least a portion of the plurality of attributes.

13. The method of claim 12, wherein the first input control provides a selection between at least a first value and a second value for the first cohort selection criteria, and wherein the first value and the second value are determined on at least the portion of the plurality of attributes.

14. The method of claim 12, wherein the user interface further includes a second input control for a second cohort selection criterion determined based on at least the portion of the plurality of attributes.

15. The method of claim 1, wherein the visual representation of at least the portion of the second dataset include at least one of a heat map, a bar graph, a pie chart, and a line graph.

16. The method of claim 1, wherein the one or more cohort selection criteria includes an endpoint definition comprising one or more of a disease diagnosis, hospitalization, and mortality.

17. The method of claim 1, wherein the one or more cohort selection criteria includes one or more inclusion criteria or exclusion criteria.

18. The method of claim 1, further comprising:
receiving, from the client device, a second user input modifying the one or more cohort selection criteria;
updating, based at least on the one or more modified cohort selection criteria, the script for accessing the first data store storing the first dataset;
executing the updated script to retrieve, from the first dataset in the first data store, a second subset of data; and
updating the second data store to include the second subset of data retrieved from the first data store.

19. The method of claim 18, wherein the second data store is updated to include the first subset of data as a first version of the second dataset and the second subset of data as a second version of the second dataset.

20. The method of claim 18, wherein the second data store is updated by at least replacing the first subset of data with the second subset of data as the second dataset.

21. The method of claim 1, further comprising:
generating the first dataset by at least querying a third data store to retrieve at least a portion of a third dataset stored therein; and
updating the first data store to include the first dataset.

22. The method of claim 21, wherein the third data store is a relational database and the first data store is a non-relational database.

23. The method of claim 22, wherein the generating of the first dataset includes transforming at least a portion of the third dataset retrieved from the third data store from a predefined schema of the relational database to a dynamic schema of the non-relational database.

24. The method of claim 21, wherein the generating of the first dataset includes performing a domain based filtering of the third dataset.

25. The method of claim 21, wherein the generating of the first dataset includes joining at least the portion of the third dataset.

26. The method of claim 1, further comprising:
receiving, from the first client device, the first user input specifying a first cohort selection criteria; and
receiving, from a second client device, a second user input modifying the first cohort selection criteria and/or specifying a second cohort selection criteria.

27. The method of claim 1, further comprising:
in response to the first user input specifying a first value for a cohort selection criterion, generating the second dataset to correspond to the first dataset retrieved from the first data store; and
in response to the first user input specifying a second value for the cohort selection criterion, generating a further subset of the first subset of the first dataset corresponding to the second value of the cohort selection criterion and generating the second dataset to correspond to the further subset of the first subset of the first dataset.

28. The method of claim 1, further comprising:
authenticating a user associated with the first client device by at least sending, to a project and user management system, a user credential information from an active directory of a secure environment and receiving, from the project and user management system, one or more client devices with access to a project associated with the first dataset.

29. A system, comprising:
at least one data processor; and
at least one memory storing instructions, which when executed by the at least one data processor, result in operations comprising:
receiving, from a first client device, a first user input specifying one or more cohort selection criteria;
generating, based at least on the one or more cohort selection criteria, a script for accessing a first data store storing a first dataset;
executing the script to retrieve, from the first dataset in the first data store, a first subset of data;
generating, for storage at a second data store, a second dataset corresponding to the first subset of data retrieved from the first data store; and
generating, for display at the first client device, a visual representation of at least a portion of the second dataset.

30. A non-transitory computer readable medium storing instructions, which when executed by at least one data processor, result in operations comprising:
receiving, from a first client device, a first user input specifying one or more cohort selection criteria;
generating, based at least on the one or more cohort selection criteria, a script for accessing a first data store storing a first dataset;
executing the script to retrieve, from the first dataset in the first data store, a first subset of data;
generating, for storage at a second data store, a second dataset corresponding to the first subset of data retrieved from the first data store; and
generating, for display at the first client device, a visual representation of at least a portion of the second dataset.
